(19) United States
(12) Patent Application Publication
(10) Pub. No.: US 2026/0232241 A1
(43) Pub. Date: Aug. 13, 2026

(54) DETERMINING AND TRACKING EMOTIONAL WELL-BEING

(71) Applicants: Gregory G Menvielle, Boulder, CO (US); Matthew So, Danville, CA (US); Robbie Vann-Adibé, Sioux Falls, SD (US)

(72) Inventors: Gregory G Menvielle, Boulder, CO (US); Matthew So, Danville, CA (US); Robbie Vann-Adibé, Sioux Falls, SD (US)

(21) Appl. No.: 19/174,737

(22) Filed: Apr. 9, 2025

Related U.S. Application Data
(60) Provisional application No. 63/757,187, filed on Feb. 11, 2025.

(51) Int. Cl. A61B 5/16; A61B 5/00; G16H 10/20; G16H 10/60; G16H 50/30; G16H 80/00

(52) U.S. Cl. CPC A61B 5/165; A61B 5/163; A61B 5/4803; A61B 5/7264; A61B 5/7275; A61B 5/741; A61B 5/744; A61B 5/746; A61B 5/7475; G16H 10/20; G16H 10/60; G16H 50/30; G16H 80/00; A61B 2562/0204

(57) ABSTRACT

A system has a computerized device with a display screen, a video camera focused to a region in front of the display screen, a microphone, a speaker, an Internet-connected server having a processor coupled to a data repository storing data and executable code, and an avatar generated by the processor and displayed on the display screen of the computerized device. The avatar questions a subject in front of the computerized device, verbal responses are transmitted as an audio stream to the processor, a video stream of the subject's face is captured by the video camera and transmitted to the processor, and the processor, executing coded instructions from the data repository, analyzes the audio stream and the video stream and results of the analysis are used to create an index value indicating a mental and emotional state of the subject.

### CROSS-REFERENCE TO RELATED APPLICATIONS

The instant application claims benefit to co-pending provisional application 63/757,187 filed Feb. 11, 2025. All disclosure of the parent application is incorporated at least by reference.

### BACKGROUND OF THE INVENTION

### Field of the Invention

The present invention is in the technical field of determining mental and fatigue states for individuals and pertains more particularly to a process of making a determination by observing and measuring features found in visual, audio, textual, and augmented (for example but not limited to geo-location or wearables) data.

### Description of Related Art

There are known in the art methods for making a determination of mental and fatigue states in regard to a person. In the United States, mental health evaluations typically rely on standardized protocols that compare an individual's symptoms to population-based norms. These assessments are conducted in a variety of settings, such as clinics, hospitals, and telehealth services, with the goal of diagnosing and monitoring mental health conditions. Most conventional diagnostic processes are guided by the well-known Diagnostic and Statistical Manual of Mental Disorders (DSM-5-TR) and the also well-known International Classification of Diseases (ICD-11). A Patient Health Questionnaire (PHQ-9) is particularly popular for screening depression due to its proven efficiency and reliability. Beyond the PHQ-9, practitioners also use tools like the Generalized Anxiety Disorder 7-item Scale (GAD-7), the Perceived Stress Scale (PSS), the Beck Depression Inventory (BDI), and the Structured Clinical Interview for DSM-5 (SCID-5) for more comprehensive evaluations.

Conventional methods at the time of filing the instant patent application for determining mental health of a person are universally based on declaration and input from the person for whom a determination is to be accomplished, often derived in a personal interview by a mental health professional or a third party supervised by a mental health professional.

What is needed in art is an artificial intelligence (AI) based system comprising video, audio and textual data regarding a subject, addition of AI analysis of the collected data, and determination of an Index that indicates the subject's mental health state, the index usable by third parties in determining support, treatment options for the subject, or to develop other novel hardware/software to further the field of mental health.

### BRIEF SUMMARY OF THE INVENTION

In one embodiment of the invention a system is provided comprising a computerized device having a display screen, a video camera focused to a region in front of the display screen, a microphone, and a speaker, an Internet-connected server having a processor coupled to a data repository storing data and executable code and a virtual assistant (VA) generated by the processor. The virtual assistant questions a subject in front of the computerized device, verbal responses are transmitted as an audio stream to the processor, a video stream of the subject's face is captured by the video camera and transmitted to the processor, and the processor, executing coded instructions from the data repository, analyzes the audio stream and the video stream and results of the analysis are used to create an index value indicating a mental and emotional state of the subject.

In one embodiment the virtual assistant is an avatar displayed to the subject in the display screen. Also, in one embodiment subjects are registered to the system, each having a username and a password for logging in to the system, and a personal profile in the data repository storing at least chronological values of the index for the subject. In one embodiment the system further comprises groups of subjects associated with organizations and enterprises with personal profiles stored associated with identity of the organizations and enterprises.

In one embodiment the system schedules and conducts interviews in series for individual subjects and records results in the subject's personal profiles. In one embodiment the audio stream is analyzed by the processor, executing algorithms from the data repository, noting at least tone, pitch, rhythmic patterns, prosodic feature variability and speech modulation, and results are compared to stored standards, determining alteration of index values. Also, in one embodiment the video stream is analyzed by the processor, executing algorithms from the data repository, noting features indicative of mental or emotional state, comprising at least blinking, rapid eye movement, direction of gaze, smiles, and frowns, and results are compared to stored standards, determining alteration of index values. Also, in one embodiment values of the index are made available to the subject and to authorized mental health professionals with permission of the subject. In one embodiment the system further comprises alerts transmitted to subjects and or to mental health professionals based on variations in value of the index for a subject. And in one embodiment the system further comprises an ability to make real-time recommendations to the subjects based on their current index, and past information about the subjects.

In another aspect of the invention a method is provided, comprising generating, by a processor of an Internet-connected server coupled to a data repository storing data and executable code, a virtual assistant (VA) configured to communicate audibly through a speaker of a computerized device having a display screen and a video camera focused to a region in front of the display screen, the method having steps for conducting an interview by the VA with a subject in front of the computerized device, transmitting verbal responses of the subject captured by the microphone in the interview as an audio stream to the processor, transmitting a video stream of at least the subject's face captured by the video camera to the processor, analyzing the audio stream and the video stream for characteristics indicative of mental and emotional state of the subject, and determining and storing an index value indicating a mental and emotional state of the subject from results of the analysis.

In one embodiment of the method the VA is an avatar displayed on the display screen of the computerized device. Also, in one embodiment the method further comprises registering subjects to the system, each having a username and a password for logging in to the system, and storing a personal profile in the data repository of at least chronological values of the index for the subject. In one embodiment the method further comprises associating groups of subjects with individual organizations and enterprises and storing personal profiles of subjects associated with identity of the organizations and enterprises. And in one embodiment the system schedules and conducts interviews in series for individual subjects and records results in subject's personal profiles.

In one embodiment the method further comprises analyzing the audio stream by the processor, executing algorithms from the data repository, noting at least tone, pitch, rhythmic patterns, prosodic feature variability and speech modulation, and comparing results to stored standards, determining alteration of index values. Also, in one embodiment the method further comprises analyzing the video stream by the processor, executing algorithms from the data repository, noting features indicative of mental or emotional state, comprising at least blinking, rapid eye movement, direction of gaze, smiles, and frowns, and comparing results to stored standards, determining alteration of index values. In one embodiment the method comprises making values of the index available to the subject and to authorized mental health professionals with permission of the subject. In one embodiment the method comprises determining and recording trends in values of the index for individual subjects. And in one embodiment the method further comprises transmitting alerts to subjects and or to mental health professionals based on variations in value of the index for a subject.

### BRIEF DESCRIPTION OF THE SEVERAL VIEWS OF THE DRAWINGS

FIG. 1 is a diagrammatical illustration of a system in an embodiment of the present invention.

FIG. 2A is a representation of a cellular telephone in use interacting with functions of the system of FIG. 1.

FIG. 2B is a representation of a view of a user through a video camera of the cellular telephone of FIG. 2A.

FIG. 3 is a representation of the view of FIG. 2B in an embodiment of the invention.

FIG. 4 is a diagrammatical representation of organization of memory in an embodiment of the invention.

### DETAILED DESCRIPTION OF THE INVENTION

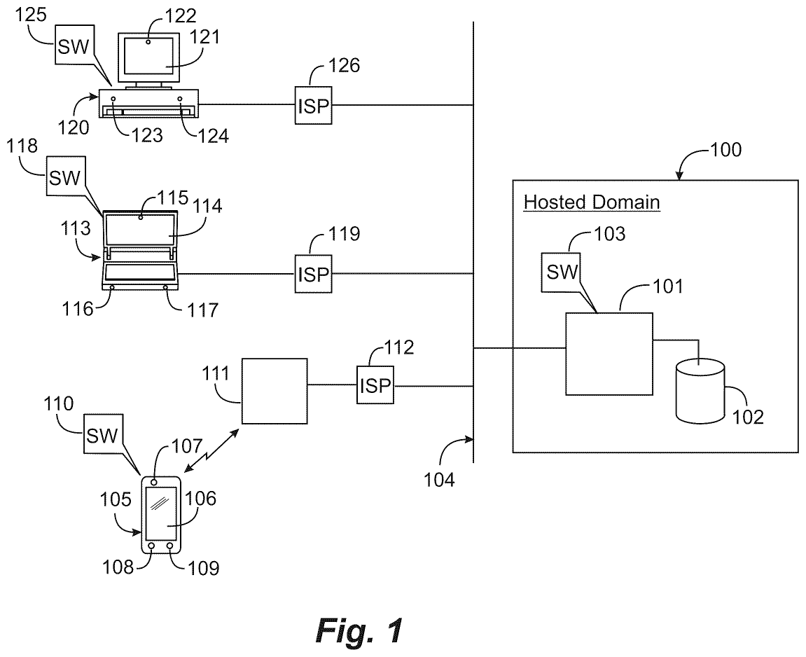

FIG. 1 is a diagrammatical illustration of a system in an embodiment of the present invention. In this system a hosted domain 100 has at least one computer processor 101 coupled to a data repository 102 adapted to store data and coded instructions, executable as software (SW) 103 by processor 101. Domain 100 is Internet connected, and line 104 represents all of the interconnected paths and subnetworks in the well-known Internet network. Users interact with domain 100 with computerized devices through the Internet network. As examples, a cellular telephone 105 which may be a smartphone, with a display 106, a video camera 107, a microphone 108 and a speaker 109 is illustrated communicating through a cellular network with a cell service provider 111 that is connected to an Internet service provider (ISP) 112 connecting to processor 101 in domain 100. Smartphone 105 has a processor that may execute a mobile application as SW 110. Connection to ISP 112 may be via a WiFi network or may be hardwired.

Further, a laptop computer 113 is illustrated with a display 114, a video camera 115, a microphone 116, and a speaker 117, executing an application as SW 118, connected to an ISP 119, which connects through Internet 104 to processor 101 in domain 100. A desktop computer 120 with a display 121, a video camera 122, a microphone 123 and a speaker 124 executes an application 125 and connects through an ISP 126 to processor 101 in domain 100. Exemplary devices 105, 113 and 120 are meant to represent a large number of computerized devices that individual subjects might use to interact with functions of domain 100 provided by SW 103.

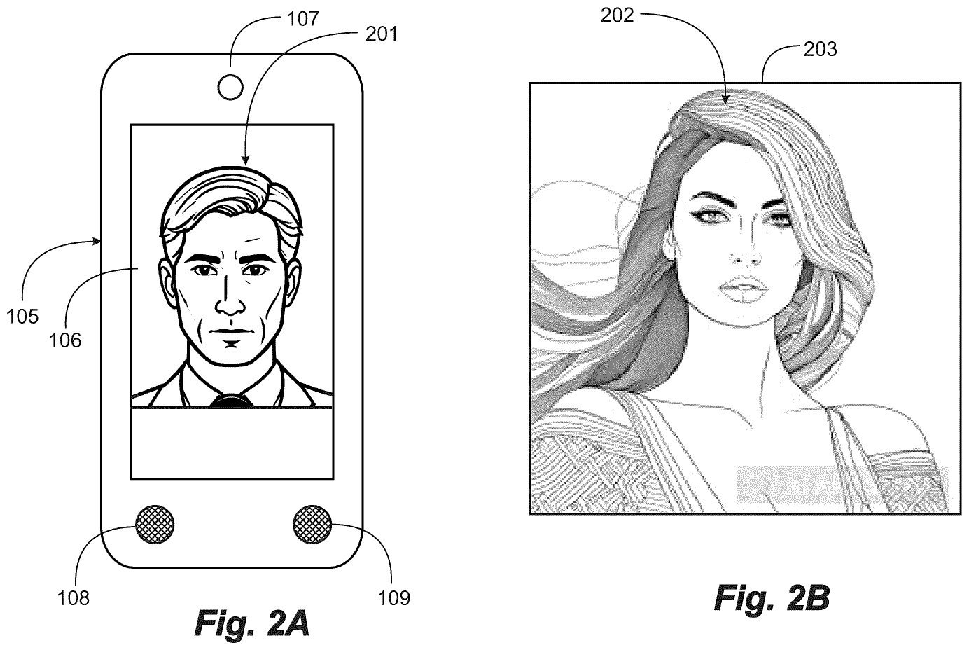

FIGS. 2A and 2B are diagrammatical representations of a subject interacting with functions of domain 100 through processor 101 executing SW 103. To interact with functions of domain 100 through processor 101 a subject user will in one embodiment open a mobile application (see SW 110, 118 and 125 in FIG. 1) and log in with a username and password. A subject user will be allotted a Username and may create a password upon navigating to domain 100 and selecting appropriate links identified for the purpose. Upon registration a portion of SW 103 will enter identity for the new user in a User Profile stored in date repository 102. The user profile for each user will store identity for the user and will store as well as interaction history, including dates and times, and retrievable recording of user interviews and interactions, including determined values of such as indexes. All this collection is also done in a way to ensure the system remains compliant with accepted security protocols such as HIPAA and GDPR guidelines. Particular care is given to ensure that the end-user is always in control of their personal information and can actively decide with whom to share this information.

A purpose of domain 100 hosted by an enterprise named herein Okaya in an embodiment of the invention is to interact with registered users in ways to determine mental and emotional health for the registered users. In one embodiment of the invention registered users interact with the Okaya domain using one of the computerized devices illustrated in FIG. 1. In one embodiment the user navigates to the Okaya site, logs in, and uses links and inputs presented in interactive interfaces by the site. In another, the user has a mobile application adapted to features of the Okaya site, and simply boots the application, which may be adapted to log in for the user and to provide links and inputs for use by the user.

In embodiments of the invention users may regularly interact and be interviewed and otherwise be examined by functions of the site. FIG. 2A illustrates cell phone 105 (see FIG. 1) in use by a registered user 202 named in this example Sandra Turner, in an interview with an avatar 201, displayed on a display screen of the cell phone in this example. It should be noted that the display of an avatar is optional in embodiments of the invention, and the system might generate a virtual assistant instead, which interacts with a voice. Following descriptions are made with an avatar.

Avatar 201 may be generated by site 100 by function of a generative AI algorithm and may or may not have a name. User Sandra Turner sees avatar 201 in display 106, hears avatar 201 through speaker 109 and may respond audibly to avatar 201 through microphone 108. Avatar 201 is displayed in this example for illustration purposes and could simply be expressed as a voice prompt rather than a full-fledged avatar.

Avatar 201 may be randomly generated, but in one embodiment the session with user Sandra Turner may be one interview of a plurality over a period of time, and the system may have determined that Sandra prefers to speak to a male avatar of a certain age range and with certain facial features, or even hair color and eye color. In such a circumstance the system may generate an avatar to the user's preference. In this circumstance the avatar may also have a name.

It is to be noted that the avatar in display 106 of the cell phone and the view in FIG. 2B 203 of user Sandra Turner taken by video camera 107 of the cell phone and transmitted to the system at domain 100 are both synced video and audio streams rather than still pics. Just a frame of each is illustrated in FIGS. 2A and 2B.

In the instant interview of user Sandra Turner avatar 201 may ask a series of questions, some of which may be generated by an AI algorithm based on previous interviews with user Turner, and some of which may be generated by user responses in the instant interview. A purpose of the questions is to elicit beliefs and opinions of the user, but also to engage the user for a time while certain other audio and visual validations may be accomplished.

Avatar 201 in one circumstance may have a series of pre-scripted questions prepared for user Turner which may be determined at least in part by previous sessions with user Turner. Questions may be for example like “How have you been since your last session? Has your mood improved?” Or more generally “Do you sleep deeply or more lightly?” There are many sorts of questions that may be presented, and the user's answers may be recorded and added to answers in previous interviews to build a base of the user's belief and opinion system. Other questions may be generated based on Turner's location, current answers, and other data provided by external captors such as, but not limited to, wearables.

The audio interaction between Turner and the Avatar are driven by an LLM model designed specifically for the application.

While user Turner is engaged with avatar 201 in questions and answers, audio assessment may be accomplished and added to stored knowledge regarding user Turner. Audio analysis has a capability to deeply evaluate speech characteristics, including vocal attributes such as tone, pitch, and rhythmic patterns. In audio assessment the system examines, but is not limited to:
1. Vocal characteristics indicative of distress or engagement
2. Prosodic feature variability
3. Speech modulation as a marker of cognitive load and emotional state

Audio technology used in the Okaya system is trained on a dataset of more than 65 demographic groups, categorized by age, ancestry, gender, and regional dialect. For all demographic groups, F1 scores for word recognition exceed 92%, which indicates high fidelity accuracy in identifying words across all demographic groups.

A linguistic analysis component examines written and transcribed speech from multiple aspects in sessions like the one described here. The system analyzes vocabulary complexity, sentence structure, and response dynamics to generate insights into cognitive and emotional states. Specific focus areas include:
1. Linguistic pattern recognition
2. Response speed assessment
3. Cognitive processing fluency indicators

It is important that conditions be optimal in a session for system functions to operate correctly and efficiently. Accordingly, the Okaya system monitors lighting and background noise to make sure the algorithm consistently “sees” and “hears” the user's inputs correctly. For instance, to optimize performance across diverse scenarios, the platform examines average grayscale pixel intensity in a video through camera 107 (instead of lux) to assess luminosity, along with image noise levels and frame rates. If there are impediments to accuracy and conditions are out of accepted ranges the avatar may ask the user to reposition a device, adjust lighting and the like to get conditions within an acceptable and operable range.

During a session the system focuses on nuanced non-verbal cues and behavioral indicators. The system analyzes facial expressions, eye movements, and other inputs, to identify signs of emotional states, including fatigue and depression. Environmental factors, such as lighting and video quality, are also considered to ensure assessment accuracy across diverse recording conditions.

Validation of the visual component involves two complementary layers of verification. First, a technical validation step aimed at validating features interpretation, uses a corpus of 324 videos to refine the system's capability to accurately identify and measure fundamental features. These features video features are collected via facial landmarks and are then transformed into facial interpretations like blinks, under varied conditions. Facial landmarks are 3D (x, y, z) coordinate locations of facial features, such as the corners of lips and eyes, points on eyebrows, irises, and face contours, and intermediate points on cheeks and forehead. This involves analyzing variables like face orientation (yaw, pitch, and roll).

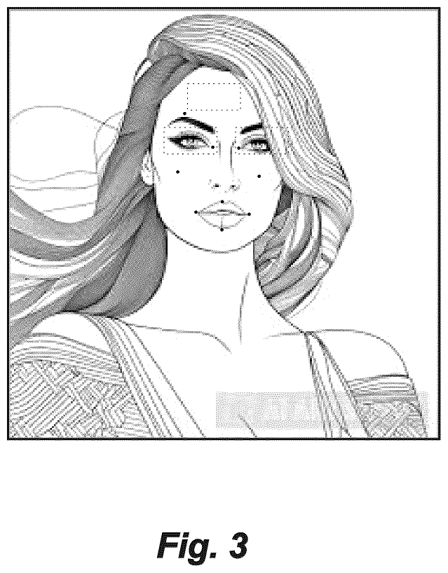

FIG. 3 represents the view of the subject as transmitted to the system by video camera 107 during an interview, in this example a view of Sandra Turner. During an interview, while the avatar asks questions the system records the subject's answers and also analyzes audio, that is language utterances by the subject while responding to the avatar. Not only are the subject's answers meaningful, but the tone, the cadence, any indication of tension in the voice, different uses of language and so on. Based on the subject's preferences, the system may have a great deal of stored samples of audio related to mental, mood, tension, fatigue and the like, and the subject's voice characteristics are compared to stored samples to help characterize the emotional state of the subject. These audio samples may come in the shape of actual recordings or simply audio-stored characteristics. The subject's nuances in verbal interaction are valuable in determining mental and emotional state.

Returning to FIG. 3 a few representative points are indicated on the subject's face, such as the corners of the mouth. While the interview proceeds, visual analysis also proceeds to capture as many clues as possible about the subject's mental state. Areas shown in dotted borders are representative of areas monitored by the system to log facial characteristics that may be compared to stored samples to gain further insight into the subject's emotional state. One area is the forehead of the subject that may be monitored for tension lines, and the state of the eyes as squinting may be monitored as well. The system also notices tics, rate of blinking, rapid eye movement, tendency to look directly or at an angle, and much more. The system stores a great deal of information relating visual nuance to mental and emotional state and fatigue.

All of the data that the system collects during a session with a user is analyzed and used to first create (after a first session) and then to update (in subsequent sessions) an Index termed here the Okaya index, as a unique score that captures a user's individual state of mental well-being and fatigue over time. Instead of one-time evaluations, it tracks personal baselines and watches for meaningful shifts in a person's wellness profile. This approach ensures insights are tailored to each person's typical range, giving more accurate, context-rich information. The Okaya Index provides a meaningful indication of a user's emotional and mental wellness and tracks changes and trends over time. On a scale of zero to 100 score categories in one example may be interpreted as follows:

| Category | Index Range | Percentile Range |
|---|---|---|
| Very Healthy | 80-100 | 65th-99th |
| Sustainable | 60-80 | 35th-65th |
| Moderate | 45-60 | 20th-35th |
| Needs Support | 0-45 | 0-20th |

The categories may be exponentially scaled to give more nuanced insights into an individual's mental well-being, with extra resolution in the lower percentiles where more support is needed. Some of the Okaya Index's utility stems from its ability to transform raw data inputs into a curated set of over 30 distinct features, which have been drawn from published scientific literature. While the index at the time of filing this patent application is still undergoing validation at scale, it is based on well-researched, multi-dimensional methodologies and is designed to offer reliable, evidence-driven insights.

In the past, organizations, individuals and enterprises have often considered mental health only as a peripheral concern. Today mental health is increasingly recognized as a core driver of both individual and organizational performance. The Okaya platform seeks to enable mental wellness in this new landscape by providing information that allows individual well-being to be monitored and, where desired, integrated into organizational goals.

While this new technology has a broad scope of potential applications, it is primarily targeted to assist personal readiness by identifying potential mental health challenges and fatigue patterns. The technology can be used to help organizations make better informed decisions about staffing and operations. Use cases range from first responders to Air Force pilots (as part of SBIR Phase II research). The system is also useful for personal health support. Clinicians and support staff simply do not have the time and resources to continuously follow all patients as they progress along their mental health journeys. This technology can be used to gain nuanced and real-time insights to assist with triage and follow-up operations. Okaya uses non-diagnostic insights such as scores and trends rather than labels, to encourage open discussions about mental health. This paradigm shifts the focus from stigma and judgment to empowerment and growth.

One important use of the Okaya system is by individuals who have a concern for their own mental well-being and may become registered users of the Okaya system to track and improve their own mental wellness. A person becomes a registered user in one embodiment by navigating to the Okaya site and registering, gaining a Username and a Password, and a personal profile which will store interactions with the individual user, create and regularly update an Okaya index for the registered user through planned and scheduled interviews, during which evaluation as described above are implemented and the index is upgraded over time, and trends may be noted as well. In one circumstance alerts may be transmitted if an index fluctuates outside of what might be considered normal variation. Particular care is given to the platform and intelligence behind the alerting system.

In other embodiments of the invention there may be functionality for the system to intervene in a situation where it is determined that a user is at a crisis, such as, for example, exhibiting indicators of suicidal inclination. The system may, in such an embodiment, have telephone numbers for first responders, police, and other agencies of intervention, associated specifically with the registered user engaged with the system.

In another important use an organization or enterprise may become a registered user, and persons associated with the organization or employed by the enterprise may become registered users and may log in for interviews on a regular basis as do individual users. In the circumstance of an organization or an enterprise regular evaluation by the Okaya system may be mandatory or a condition of employment. In some circumstances regular evaluation may be voluntary. Enterprises may use the membership with Okaya or a similar host in employee management. The inventors believe association and interaction with Okaya is in itself an inducement for persons to pay closer attention than otherwise to their mental and emotional wellness.

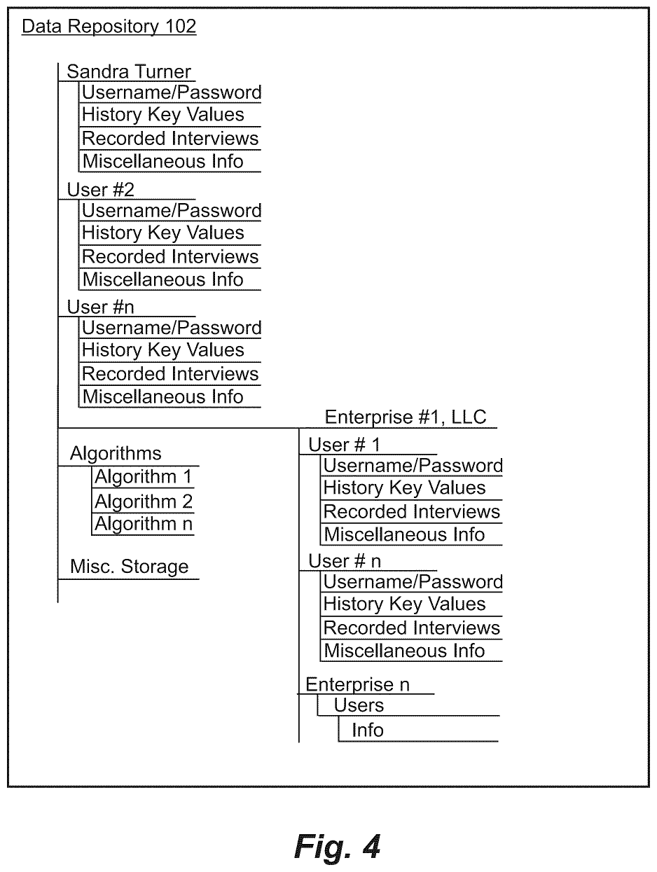

FIG. 4 is a diagrammatical representation of memory organization in data repository 102 (see FIG. 1) in an embodiment of the invention. As described briefly above, there may be records for individual registered users, who are motivated to engage with the host, in this case Okaya, to monitor and improve their mental and emotional health and well being. Records for individual members Sandra Turner, a member #2 and a member #n or indicated, which represents any number of individual registered members. Records for each individual member may be such as Username and Password, a history of Key values, which may include dates of establishment and change, trends, and alerts and the like, recorded interviews, and miscellaneous info, which may include textual information regarding a member.

In addition to records for individual registered users there may also be records for organizations and enterprises. An enterprise #1, LLC is indicated, and for enterprises and organizations records may be kept for associates of organizations and employees of enterprises. Records for example for employees of an enterprise may be very similar to records for individual registered users.

Data storage for the system may also comprise any number of algorithms as indicated in FIG. 4 as algorithms 1, 2 and n. Accessible algorithms may comprise AI programs of different sorts, such as generative AI for forming the avatar described in reference to FIG. 2A, and other executable code for other functions.

In some embodiments there may be functions for a registered user to access and review, but not to edit or alter, records for that registered user. An individual, for example, may want to review a previous interview or review the history of key values. The system puts a deliberate emphasis on privacy, security, and data ownership to preserve the proper information silos.

In the case of an organization or an enterprise there may be functionality for securely authorized users to review records stored for individual associates or employees. An official of a department of Human Resources for an enterprise may have access to records for individual employees, for example.

Another important use of the new technology described in embodiments of this invention is by mental health organizations and practitioners. In a mental health clinic, for example, clinicians and support staff may not have the time and resources to continuously follow all patients as they progress along their mental health journeys. This new technology can be used to gain nuanced and real-time insights to assist with triage and follow-up operations. A mental health clinic may associate with Okaya, for example, and use the functions of the interview process and key creation and updating to track mental and emotional health for patients of the clinic. The same may be true for mental hospitals and mental departments in hospitals.

The new technology is entirely accessible via an API creating a major breakthrough for organizations that would want to use this platform to build better tools and functionalities geared towards participants and enterprises. The Okaya API follows the same standards as the platform when it comes to security and privacy. The API can also be used to access information stored in external systems.

The Okaya system has instituted functionality to ensure ethical use of data and transparency. All data collected during Okaya-led studies are kept strictly confidential and used solely for research purposes. All data is encrypted and securely stored with access restricted to only persons thoroughly vetted and authorized to access. Participants may be provided with anonymous email addresses to protect identities. Any personal information recorded during studies or interviews is securely stored and may be deleted when not of use. Participants are asked for a very limited set of personal information.

The Okaya system is not a fixed and rigid system but is undergoing continuous development and improvement through internal studies and experimentation. Research is focused on developing more nuanced analytical capabilities to enrich understanding of mental well-being. By mapping emotional states, cognitive processes and psychological resilience with a goal to detect more subtle indicators of stress, anxiety and cognitive performance that surpass the limits of current assessment models at the time of filing this patent application. A goal is to develop a richer, more dynamic view of individual mental and emotional well-being. A central priority of the Okaya system is to enhance the process of collecting, integrating and understanding visual, auditory and textual data streams. A priority is also to better understand how mental and emotional health evolves over time. Wellness studies using collected impersonal data may track wellness trajectories over extended periods.

The Okaya system has made a commitment to upholding transparency and scientific rigor in every stage of operation, development and research. This may be accomplished by establishing collaborative research partnerships, submitting results to peer-reviewed scientific journals, incorporating systematic user feedback, and continuously refining the wellness index based on emerging insights. The system aims to advance the field of mental health assessment.

The skilled person will understand that the embodiments of apparatus and methods described in this specification are entirely exemplary and are in no way limiting to the scope of the invention. There will be, within the scope of the invention, many equivalent pieces of apparatus and functions that have not been described in detail herein but are nevertheless within the scope of the invention. The scope of the invention is limited only by the claims that follow.

1. A system, comprising:
a computerized device having a display screen, a video camera focused to a region in front of the display screen, a microphone, and a speaker;
an Internet-connected server having a processor coupled to a data repository storing data and executable code; and
a virtual assistant (VA) generated by the processor;
wherein the virtual assistant questions a subject in front of the computerized device, verbal responses are transmitted as an audio stream to the processor, a video stream of the subject's face is captured by the video camera and transmitted to the processor, and the processor, executing coded instructions from the data repository, analyzes the audio stream and the video stream and results of the analysis are used to create an index value indicating a mental and emotional state of the subject.

2. The system of claim 1 wherein the virtual assistant is an avatar displayed to the subject in the display screen.

3. The system of claim 1 wherein subjects are registered to the system, each having a username and a password for logging in to the system, and a personal profile in the data repository storing at least chronological values of the index for the subject.

4. The system of claim 3 further comprising groups of subjects associated with organizations and enterprises with personal profiles stored associated with identity of the organizations and enterprises.

5. The system of claim 3 wherein the system schedules and conducts interviews in series for individual subjects and records results in the subject's personal profiles.

6. The system of claim 1 wherein the audio stream is analyzed by the processor, executing algorithms from the data repository, noting at least tone, pitch, rhythmic patterns, prosodic feature variability and speech modulation, and results are compared to stored standards, determining alteration of index values.

7. The system of claim 1 wherein the video stream is analyzed by the processor, executing algorithms from the data repository, noting features indicative of mental or emotional state, comprising at least blinking, rapid eye movement, direction of gaze, smiles, and frowns, and results are compared to stored standards, determining alteration of index values.

8. The system of claim 1 wherein values of the index are made available to the subject and to authorized mental health professionals with permission of the subject.

9. The system of claim 1 further comprising alerts transmitted to subjects and or to mental health professionals based on variations in value of the index for a subject.

10. The system of claim 1 further comprising an ability to make real-time recommendations to the subjects based on their current index, and past information about the subjects.

11. A method, comprising:
generating, by a processor of an Internet-connected server coupled to a data repository storing data and executable code, a virtual assistant (VA) configured to communicate audibly through a speaker of a computerized device having a display screen and a video camera focused to a region in front of the display screen;
conducting an interview by the VA with a subject in front of the computerized device;
transmitting verbal responses of the subject captured by the microphone in the interview as an audio stream to the processor;
transmitting a video stream of at least the subject's face captured by the video camera to the processor;
analyzing the audio stream and the video stream for characteristics indicative of mental and emotional state of the subject; and
determining and storing an index value indicating a mental and emotional state of the subject from results of the analysis.

12. The method of claim 11 wherein the VA is an avatar displayed on the display screen of the computerized device.

13. The method of claim 11 further comprising registering subjects to the system, each having a username and a password for logging in to the system, and storing a personal profile in the data repository of at least chronological values of the index for the subject.

14. The method of claim 13 further comprising associating groups of subjects with individual organizations and enterprises and storing personal profiles of subjects associated with identity of the organizations and enterprises.

15. The method of claim 11 wherein the system schedules and conducts interviews in series for individual subjects and records results in subject's personal profiles.

16. The method of claim 11 further comprising analyzing the audio stream by the processor, executing algorithms from the data repository, noting at least tone, pitch, rhythmic patterns, prosodic feature variability and speech modulation, and comparing results to stored standards, determining alteration of index values.

17. The method of claim 11 further comprising analyzing the video stream by the processor, executing algorithms from the data repository, noting features indicative of mental or emotional state, comprising at least blinking, rapid eye movement, direction of gaze, smiles, and frowns, and comparing results to stored standards, determining alteration of index values.

18. The method of claim 11 comprising making values of the index available to the subject and to authorized mental health professionals with permission of the subject.

19. The method of claim 11 comprising determining and recording trends in values of the index for individual subjects.

20. The method of claim 11 further comprising transmitting alerts to subjects and or to mental health professionals based on variations in value of the index for a subject.
